(19) United States
(12) Patent Application Publication
(10) Pub. No.: US 2019/0274599 A1
(43) Pub. Date: Sep. 12, 2019

(54) In Vivo Extraction of Interstitial Fluid Using Hollow Microneedles

(71) Applicants: National Technology & Engineering Solutions of Sandia, LLC, Albuquerque, NM (US); STC. UNM, Albuquerque, NM (US)

(72) Inventors: Ronen Polsky, Albuquerque, NM (US); Philip Rocco Miller, Albuquerque, NM (US); Justin T. Baca, Albuquerque, NM (US)

(21) Appl. No.: 16/284,890

(22) Filed: Feb. 25, 2019

Related U.S. Application Data
(62) Division of application No. 15/078,870, filed on Mar. 23, 2016, now abandoned.
(60) Provisional application No. 62/144,545, filed on Apr. 8, 2015.

(51) Int. Cl. A61B 5/145

(52) U.S. Cl. CPC A61B 5/14514; A61B 5/1468; A61B 5/14546

(57) ABSTRACT

A transdermal and/or intradermal diagnostic device comprising a combined hollow microneedle interstitial fluid (IF) extraction device and a detector can monitor biomarkers in-situ. For example, electrode transducers with optimally arrayed and designed microneedles can be combined with a suitable pumping method to determine biomarker levels in human subjects under intense physical exertion to monitor metabolic stress and fatigue. The device can perform real-time, in-situ measurements of lactate in human subjects. Monitoring of other biomarkers is straightforward.

### CROSS-REFERENCE TO RELATED APPLICATIONS

This is a divisional application of U.S. application Ser. No. 15/078,870, filed Mar. 23, 2016, which claims the benefit of U.S. Provisional Application No. 62/144,545, filed Apr. 8, 2015, both of which are incorporated herein by reference.

### STATEMENT OF GOVERNMENT INTEREST

This invention was made with Government support under Contract No. DE-NA0003525 awarded by the United States Department of Energy/National Nuclear Security Administration. The Government has certain rights in the invention.

### FIELD OF THE INVENTION

The present invention relates to metabolic and health monitoring of humans and, in particular, to a transdermal and/or intradermal diagnostic device for in-vivo extraction of interstitial fluid using hollow microneedles.

### BACKGROUND OF THE INVENTION

The threat of exposure to chemical and biological agents facing the warfighter makes fast and accurate analysis of health and physiology of particular importance, especially when direct detection of known agents is not feasible. In addition, the ability to quickly and accurately monitor the health of an individual without the necessity of a medical specialist would be invaluable for personalized healthcare and decentralized testing capabilities. In these situations, the ability to assess the immediate physiological state of an individual is a valuable indicator to achieve situational awareness and determine whether medical counter measures are necessary. In addition, the ability to autonomously monitor human pathophysiology in real time can help determine the limits of human performance such as electrolyte deficiencies and extended periods of physical stress and would be advantageous for general sports fitness and home healthcare applications. Even for elite athletes, overexertion can result in impaired performance for weeks up to years. See F. B. Wyatt et al., “The Overtraining Syndrome: A Meta-Analytic Review,” Journal of Exercise Physiology Online 16(2), (2013). However, little data is available concerning tissue levels of biomarkers of metabolic stress for deployed military personnel, elite athletes, and fatigued humans, generally.

Exercise physiology studies as well as studies looking at biomarkers in the blood of military personnel undergoing intense training exercises have identified cortisol, glutamine, glutamate, serum lactate, interleukin-6, testosterone, thyroid hormones, human growth hormone, insulin and glucose, adrenaline, and neuropeptide Y (NPY) as important biomarkers for overtraining syndrome and fatigue. See D. Purvis et al., “Physiological and psychological fatigue in extreme conditions: overtraining and elite athletes,” PM&R 2(5), 442 (2010); X. Li et al., “Experimental study on neuroendocrinological and immunological characteristics of the military-trained artillerymen,” Chinese medical journal 125(7), 1292 (2012); and S. R. Weeks et al., “Physiological and psychological fatigue in extreme conditions: the military example,” PM&R 2(5), 438 (2010). Fatigue associated with combat simulations also results in increased resting levels of oxygen consumption and increased production of adrenaline and NPY. NPY increases adrenaline production, decreases anxiety and enhances memory and attention. The combination of adrenaline and NPY production enhances performance, even under stress conditions. See S. R. Weeks et al., “Physiological and psychological fatigue in extreme conditions; the military example,” PM&R 2(5), 438 (2010). However, it is unknown how long this high level of performance can be maintained. It is noteworthy that military personnel receiving uncharacteristically rigorous training, such as Special Forces, return to basal levels of biomarkers much more quickly than their peers who have not had the same level of training.

Many conventional diagnostic monitoring methods rely on macroscale systems that are undesirable due to requirements for large sample volumes, user operation, fluid transfer between components, and the pain/tissue damage that can result from long-term device/human interactions. Microneedle-enabled analysis systems offer an ideal solution to these problems. Their size enables minimally-invasive interrogation of interstitial fluid (IF) due to their ability to puncture the epidermis with minimal irritation of dermal layers of the skin associated with pain, blood flow, and sensation. The predominant use of previously described microneedles has been for drug delivery, and there has been little research on the use of microneedles for minimally invasive point-of-care sensing. Additionally, no current microneedle platform is capable of performing long term sensing of multiple biomarkers.

Methods exist for using microneedles to enable analyte detection that usually fall within one of the three following methods: extraction of fluid (IF or blood) for off-body analysis, in vivo detection using microneedles as electrodes, or using the microneedles as probes for capturing and extracting circulating entities to be analyzed ex-vivo. See E. V. Mukerjee et al., “Microneedle array for transdermal biological fluid extraction and in situ analysis,” Sensors and Actuators A: Physical 114(2), 267 (2004); P. R. Miller et al., Biomicrofluidics 5, 013415 (2011); and S. R. Corrie et al., “Surface-modified microprojection arrays for intradermal biomarker capture, with low non-specific protein binding,” Lab on a Chip 10(20), 2655 (2010). Using microneedles as probes for collection of circulating biomolecules or as electrodes themselves is a facile method for acquiring information regarding the health of an individual; however this style of device is not amenable for on-body detection. See S. R. Corrie et al., “Surface-modified microprojection arrays for intradermal biomarker capture, with low non-specific protein binding,” Lab on a Chip 10(20), 2655 (2010). Removal of devices from skin is necessary for analyzing captured biomarkers since multiple incubation and washing steps are necessary in order to generate a signal. Microneedles with biosensors built into their surface work well for single tests or over short periods of time, however fouling tarnishes their signal and pore closure prohibits long-term detection. See J. R. Windmiller et al., “Bicomponent Microneedle Array Biosensor for Minimally-Invasive Glutamate Monitoring,” Electroanalysis 23(10), 2302 (2011); and M. A. Invernale et al., “Microneedle Electrodes Toward an Amperometric Glucose-Sensing Smart Patch,” Advanced healthcare materials (2013). Microneedle-based systems that extract fluid can be paired with microfluidic chips to enable detection of more sophisticated analytes (e.g. proteins, viruses) to create an on-body detection platform. Previous groups have studied this integration of microneedles and microfluidics, however, some of the initial studies lacked the sophistication of the sensing component. Mukerjee et al. used hollow microneedle arrays made with standard silicon microfabrication techniques to extract IF from a human subject and detect glucose on-chip using a commercially available colorimetric glucose strip. See E. V. Mukerjee et al., Microneedle array for transdermal biological fluid extraction and in situ analysis,” Sensors and Actuators A: Physical 114(2), 267 (2004). While this study showed the ability to extract fluid from a human subject, the detection strips used were not reusable and required the user to continuously replace the detection strip and manually monitor each reading.

Currently, no autonomous and portable diagnostic platforms are available for remote metabolic monitoring. Therefore, a need exists for a transdermal diagnostic device that is autonomous, portable, robust, and provides an ability to readily monitor individuals, especially in extreme environments, and for personalized healthcare.

### SUMMARY OF THE INVENTION

The present invention is directed to a transdermal and/or intradermal diagnostic device for monitoring ones immediate pathophysiological state, metabolic stress and fatigue in a human, comprising a single or an array of hollow microneedles adapted to penetrating the skin of the human, wherein the skin comprises at least one biomarker in an interstitial fluid; and a microfluidic chip adapted to extract the interstitial fluid through the hollow microneedles and collect the at least one biomarker in a sample reservoir in the microfluidic chip. For example, the at least one biomarker can comprise a biomarker for metabolic stress or fatigue, such as cortisol, a ketone, TNF-α, glutamine, glutamate, interleukin-6, testosterone, thyroid hormone, human growth hormone, insulin, glucose, adrenaline, neuropeptide Y, or lactate. The concentration of the at least one biomarker in the interstitial fluid preferably correlates with the concentration of the at least one biomarker in the blood plasma of the human. The device can further comprise a spectrophotometer for analyzing the at least one biomarker in the extracted interstitial fluid. The device can further comprise an electrode transducer for sensing the at least one biomarker in the extracted interstitial fluid. Preferably, the hollow microneedles have a bore opening in the middle third on the side of each microneedle. The hollow microneedles preferably have an aspect ratio between 2 and 5, and a base of between 300 and 500 microns. The hollow microneedles can further comprise a coating to control hydrophilicity and promote fluid flow through the lumen of the microneedle. The microfluidic chip can further comprise a pump for sucking the biomarker-containing interstitial fluid into the sample reservoir. For example, the pump can be a vacuum pump, a capillary force pump, a microdialysis pump, or a pulsatile vacuum pump. The microfluidic chip can further comprise an injector for injecting saline solution into the skin through the array of hollow microneedles.

The invention is also directed to a method for extracting interstitial fluid from a human or an animal, comprising providing an interstitial fluid extraction device and extracting the interstitial fluid through the array of hollow microneedles and collecting the at least one biomarker in the sample reservoir in the microfluidic chip. The extraction device can comprise a vacuum pump and the step of extracting can comprise sucking the interstitial fluid through the hollow microneedles into the sample reservoir. The method can comprise injecting a saline solution into the skin through the hollow microneedles to mix with the interstitial fluid and extracting the mixed saline solution back out through the hollow microneedles into the sample reservoir. The method can comprise filling the sample reservoir with a saline solution and diffusing the at least one biomarker from the interstitial fluid through the hollow microneedles into the saline solution in the sample reservoir.

### BRIEF DESCRIPTION OF THE DRAWINGS

The detailed description will refer to the following drawings, wherein like elements are referred to by like numbers.

FIG. 1(a) is a photograph of a nine-element microneedle array in plastic laminate fluidic manifold. FIG. 1(b) is a photograph of a hollow microneedle.

FIG. 2 is a schematic illustration of the steps of an additive process to fabricate a microneedle structure. A similar process was used to fabricate the hollow microneedle shown in FIG. 1(b).

FIGS. 3(a) and 3(b) are schematic illustrations of a vacuum-assisted IF extraction device. FIG. 3(a) shows the device prior to extraction. FIG. 3(b) shows the device after extraction wherein a vacuum pump is used to suck interstitial fluid through a hollow microneedle array into a sample reservoir.

FIGS. 4(a) and 4(b) are schematic illustration microdialysis-inspired IF extraction device. FIG. 4(a) shows the device prior to extraction wherein a saline solution is injected into the skin through a hollow microneedle array. FIG. 4(b) shows the device after extraction wherein a vacuum pump is used to suck the injected saline solution back into a sample reservoir.

FIGS. 5(a) and 5(b) are schematic illustrations of a diffusion-assisted biomarker extraction device. FIG. 5(a) shows the device prior to extraction wherein a sample reservoir is prefilled with saline solution. FIG. 5(b) shows the device after extraction wherein biomarker analytes in the interstitial fluid are extracted by diffusion through a hollow microneedle array into the saline-filled sample reservoir.

FIG. 6 is a photograph of an exemplary microneedle-based extraction system worn on a forearm. The tubing connects to a vacuum source.

### DETAILED DESCRIPTION OF THE INVENTION

The present invention is directed to the use of microneedles to transdermally access biomarkers for monitoring the exposure of humans to chemical and biological weapons, overexertion in athletes, and fatigue in humans, and for general healthcare. According to the invention, needle geometries are provided that are best suited to penetrate the skin and extract adequate quantities of IF with minimal discomfort. Biomarkers of stress and fatigue are used that are accessible in the IF at concentration levels that correlate with clinically-relevant blood/plasma levels. Sensor transducers that can measure biomarkers of stress and fatigue using lactate as a surrogate system are described as an example of the invention. The invention enables a wearable, transdermal diagnostic device capable of interfacing with a warfighter, athlete, or other human in the field and allows for realtime and remote physiological monitoring of exposure to chemical, biological, radiological, and nuclear (CBRN) agents, or the buildup of indicators of stress or fatigue. The present invention can be used as both a training tool as well as an important asset to help determine the health status of a warfighter or athlete realtime and thereby improve human performance and general health monitoring.

### Optimal Microneedle Geometries for Extracting IF In-Vivo while Minimizing Discomfort

The optimal hollow microneedle geometry for extracting interstitial fluid was not been heretofore known. Previous groups have investigated the effect of microneedle bore location on the microneedle for IF extraction; however, bore placement within dermal tissue has not been studied in vivo. There are seven histological layers of the combined epidermis and dermis of the skin. Fluid concentration and accessibility will vary across the seven layers, thus, microneedle length and bore placement can have a profound influence on the amount of fluid that can be extracted. In particular, the placement of the needle bore opening and the aspect ratio of the microneedles are critical components in optimizing extraction rates of interstitial fluid.

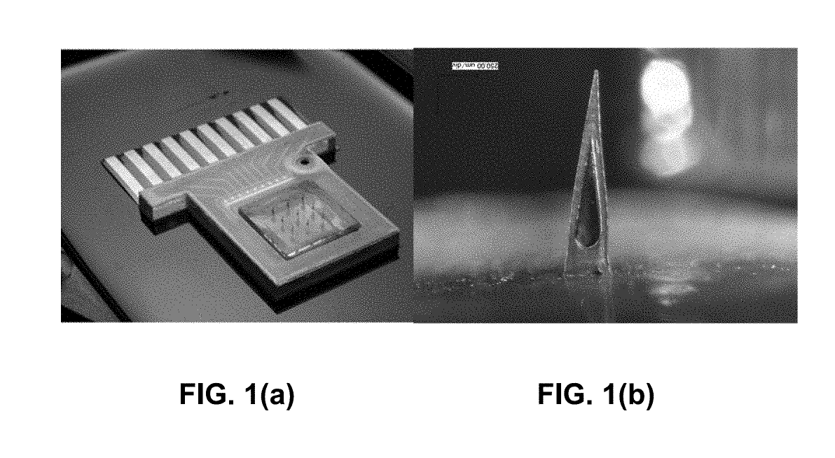

The flow of IF can be influenced by possible tissue occlusion of the microneedle bore. This can be mitigated by placing the needle bore on the side of the needle to prevent coring within the microneedle bore. The placement of the bore opening on the side of the microneedle rather than the tip avoids the known problem of tissue occlusion and increases the flow of extracted IF in vivo. More preferably, the placement of the bore opening on the side of the middle third of the microneedle as opposed to the base or tip of the microneedle optimizes the flow of extracted IF in vivo. For example, pyramidal microneedles have been designed to avoid tissue occlusion using side bore placement, as shown in FIG. 1(b). An aspect ratio of between 2 and 5 is optimum for extracting IF and avoiding microneedle fracturing upon insertion. For example, microneedles can be fabricated with an aspect ratio of 3 and a base of 300 to 500 microns.

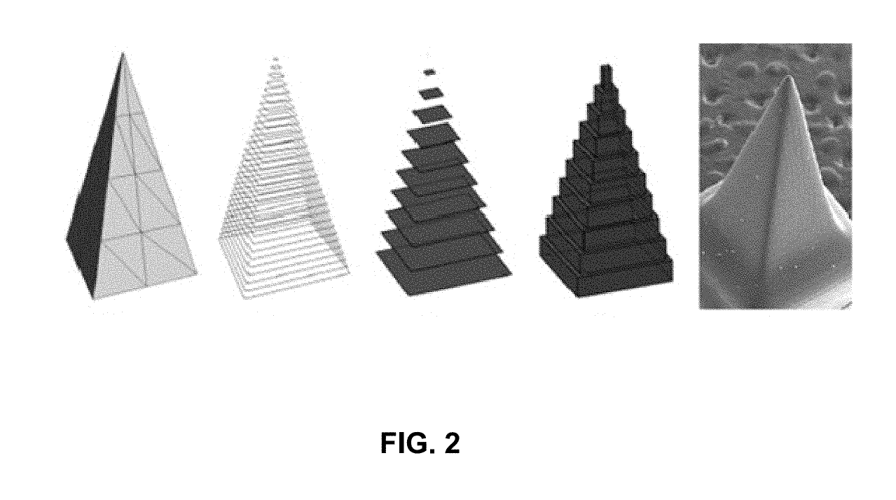

A micron-scale three-dimensional (3D) additive fabrication technique can be used to overcome limitations of traditional needle fabrication methods. Two-photon polymerization involves near simultaneous absorption of ultrashort laser pulses for selective curing of photosensitive material and is a powerful tool to control microneedle geometry. See R. J. Narayan et al., “Medical prototyping using two photon polymerization,” Materials Today 13(12), 42 (2010). The result is a rapid prototyping system that can fabricate complex 3D structures without a mask based on a 3D computer-aided design (CAD) model, as shown in FIG. 2. This allows for nearly unlimited user control of the fabricated parts with resolution down to sub-micron levels with commercially available, biocompatible photoresists with quick turnaround. The dexterity of this fabrication technique allows altering the placement of the bore with high precision and adjusting any other dimension of the microneedle in order to optimize the geometry for IF extraction.

Typically, only small volumes (1-10 μl) of IF can be extracted using a single microneedle. Microneedle arrays increase the volume and speed of IF extraction compared to individual needles. Using results from the optimization of single needle geometries, the effects of microneedle array size and needle spacing can be determined. While the number of needles is expected to increase the extracted fluid volume, there is not necessarily a linear relationship between the number of needles and total IF volume extracted. An optimal microneedle spacing allows complete penetration of individual needles into skin, minimizes discomfort, and maximizes IF extraction. A change in puncture mechanics when using arrayed microneedles can also affect optimal microneedle spacing. See A. Davidson et al., “Transdermal drug delivery by coated microneedles: geometry effects on effective skin thickness and drug permeability,” Chemical Engineering Research and Design 86(11), 1196 (2008). The distance between microneedles relative to microneedle height can be optimized such that puncture sites exist for each needle on the array and the depth of each insertion compares to results seen in the single microneedle studies. Previous studies have shown closely spaced needles do not act as individual needles when inserted in the skin and suffer from “tenting,” causing the skin to stretch around the needle but not puncture. See O. Olatunji et al., “Influence of array interspacing on the force required for successful microneedle skin penetration: Theoretical and practical approaches,” Journal of pharmaceutical sciences 102(4), 1209 (2013). Puncture sites for all needles in an array can be confirmed in ex vivo porcine skin prior to validation in a human study. In addition to optimal microneedle geometry, array spacing, and extraction method, other design choices can be optimized. These include using particular microneedle coatings to control hydrophilicity and further promote fluid flow through the lumen of the microneedle and applying pressure to the skin surface to be accessed by the needle.

Different “pumping” methods can be used for IF extraction. Methods for extracting IF include vacuum suction, capillary force wicking, pulsatile vacuum extraction, microdialysis, and diffusion. These techniques can be directly compared in vivo in terms of IF volume extracted, IF rate of extraction, and the feasibility of incorporating the method of extraction with an on-body device. Systematic requirements (e.g. power, pumps, and valves) necessary for an integrated analysis system based on microneedle extraction of IF can be determined for each extraction method.

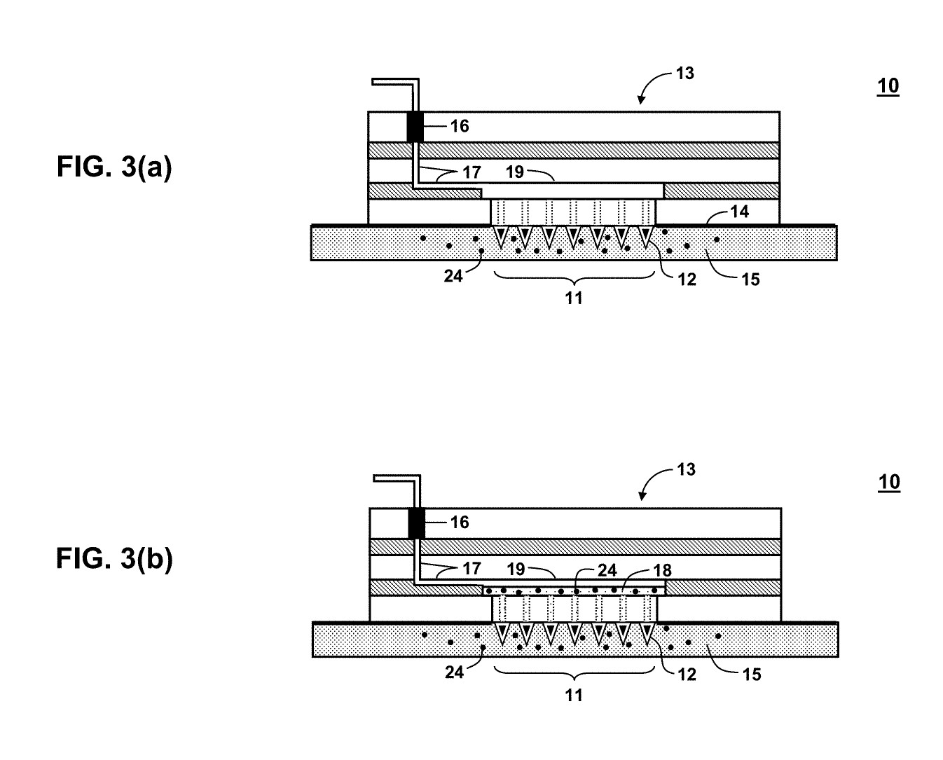

Negative-pressure-assisted (vacuum) extraction can be used to access IF through a hollow microneedle array. FIGS. 3(a) and 3(b) show a vacuum-assisted IF extraction device 10. The device 10 comprises an array 11 of hollow microneedles 12 supported by a microfluidic chip 13. The hollow microneedle array 11 can penetrate the skin 14 for access to the biomarker-containing IF 15. A vacuum pump 16 and associated fluidic channels 17 can be used to extract the IF 15 and biomarkers 24 through the hollow microneedle array 11. For example, negative pressure can be applied with a syringe pump to the microneedle array to enhance IF extraction. See P. M. Wang et al., “Minimally invasive extraction of dermal interstitial fluid for glucose monitoring using microneedles,” Diabetes technology & therapeutics 7(1), 131 (2005). Alternatively, a simply capillary force method can be used to wick the IF through the hollow microneedles. The extracted fluid 18 can be collected in a microfluidic sample reservoir 19 on the chip 13. Flow rates can be adjusted to minimize bore occlusion in the microneedles due to suction of tissue. While the negative pressure method has shown some success, alternative IF extraction techniques can be more amenable to an on-body diagnostic platform due to power and spacing requirements of such a device.

Pulsatile vacuum extraction of IF can be more efficient than continuous or capillary force extraction. Pulsatile negative pressure can be superior because it allows interstitial fluid to intermittently refill around the dermal locations where the needles reside between vacuum pulses. This intermittent negative pressure can decrease problems of tissue occlusion of the needle bores and enhance IF extraction. Further, the pulsatile vacuum extraction is painless, and well-tolerated by human subjects.

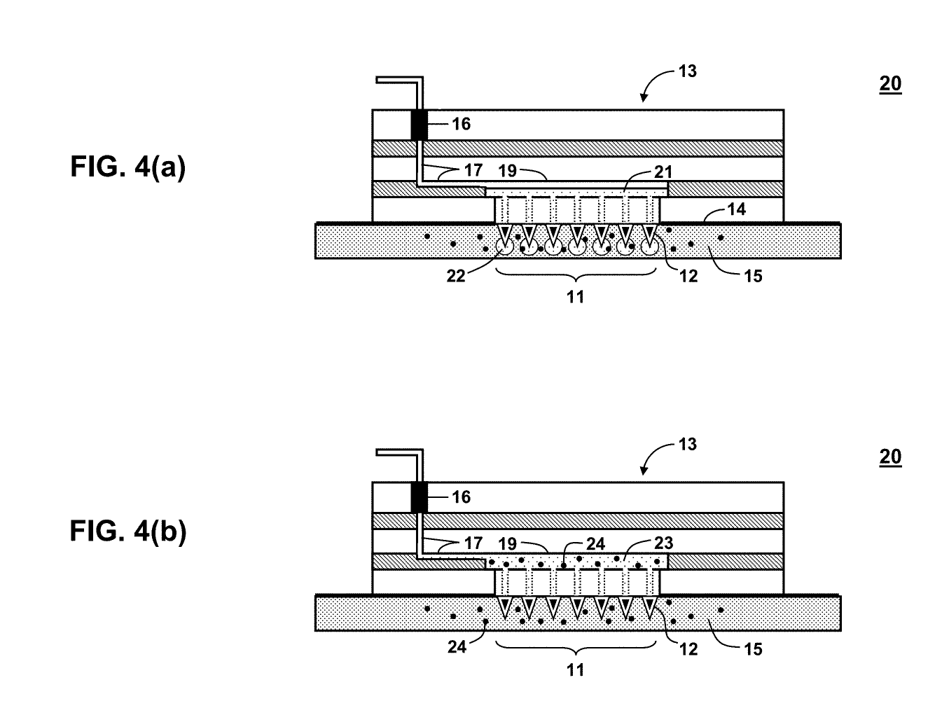

A microdialysis-inspired device 20 can be used wherein saline solution 21 is injected 22 into the skin 14 through the hollow microneedle array 11 to mix with the IF 15 and then retrieved with the mixed biomarkers 24 back through the array 11 via negative pressure from a pump 16, as shown in FIGS. 4(a) and 4(b). This method can be used when the skin 14 may be compressed during microneedle insertion such that rapid relaxation of the tissue and refilling of dermal layers with IF is not possible with a pulsatile method. This dermal compression effect has been seen with drug delivery studies with microneedles and causes increased fluidic resistance which minimizes the amount of fluid that can be delivered. See W. Martanto et al., “Mechanism of fluid infusion during microneedle insertion and retraction,” Journal of controlled release 112(3), 357 (2006). A brief settling time can be allowed after saline injection prior to extraction so that extracted fluid 23 is not significantly diluted in biomarkers 24 relative to analyte concentrations in the IF 15.

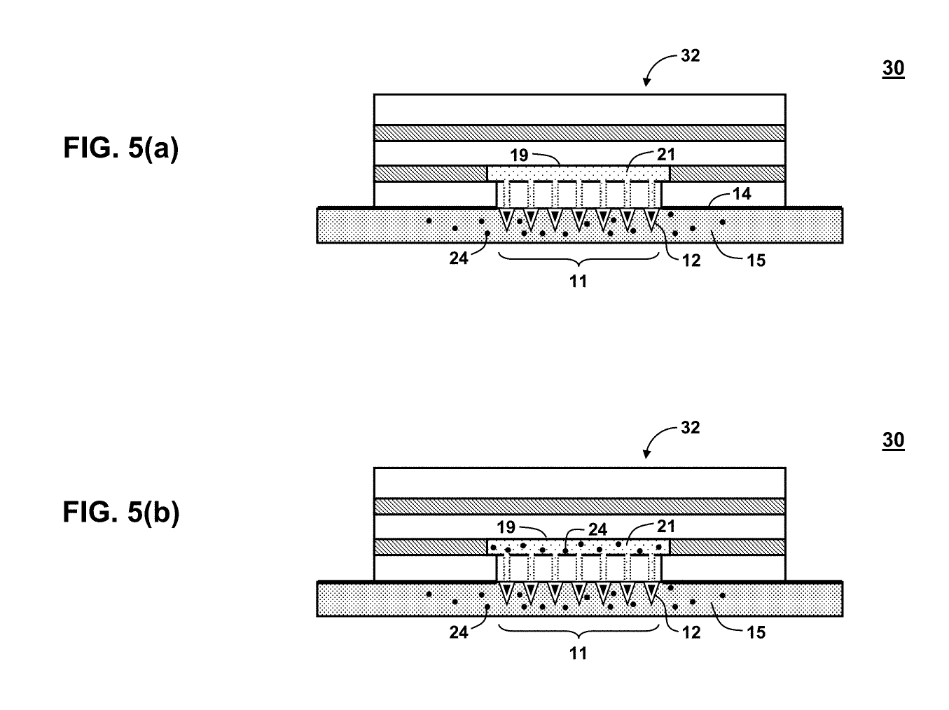

A passive, diffusion-assisted device 30 for analyte extraction based on IF equilibration with an internal saline reservoir can also be used, as shown in FIGS. 5(a) and 5(b). The sample reservoir 19 can be prefilled with a saline solution 21 before applying the hollow microneedle array 11 to the skin 14. Once the microneedles 12 are within the dermal tissue, biomarker analytes 24 within the IF 15 equilibrate with the saline solution 21 in the sample reservoir 19 on the microfluidic chip 32. While this method is not as efficient for IF extraction as vacuum methods, it can eliminate the need for pumping and can provide a simplified sensing platform.

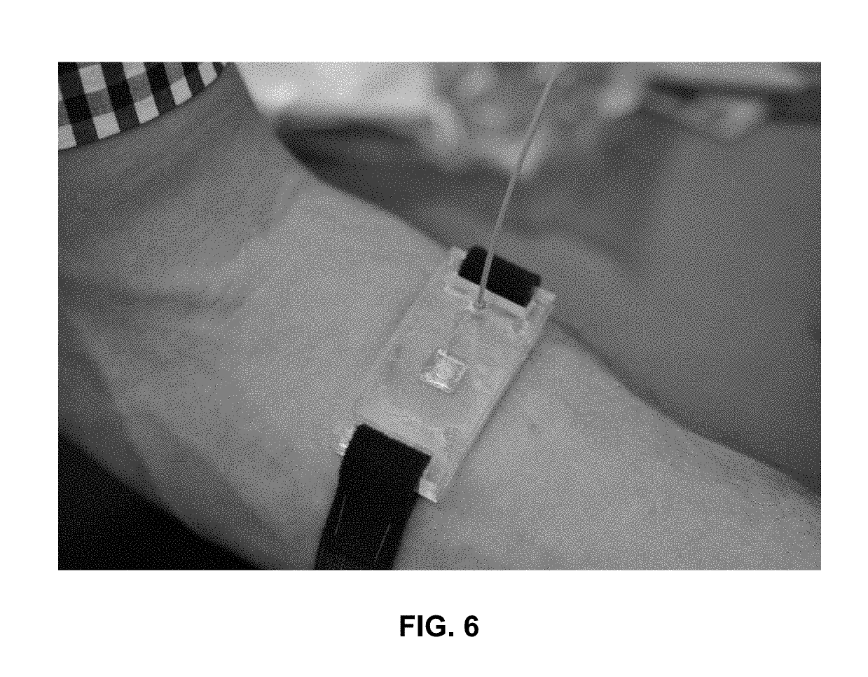

The microneedles can be mounted on a microfluidic chip and attached to a syringe assembly through sterile tubing. The microfluidic chip can be used to secure the microneedle, and allows for a total insertion depth of up to 2 mm. For example, the microneedles with attached syringe can be used to extract IF from the mid forearm, as shown in FIG. 6. For example, the microneedles can remain in place for 10-20 minutes for collection of sufficient interstitial fluid from the forearm.

### Identification of Stress/Fatigue Biomarkers that are Extractable from IF Using Microneedles and Correlation of Interstitial Levels with Known, Clinically-Relevant Blood/Plasma Levels that are Indicative of Metabolic Stress or Fatigue

Interstitial fluid contents and biomarker concentrations remain incompletely characterized. These biomarkers can correlate with commonly measured plasma levels during conditions of stress or fatigue. Therefore, the correlation between serum and IF biomarker composition can be determined. The concentration of known markers of metabolic stress and fatigue (e.g., lactate, glucose, ketones, cortisol, and TNF-α) in extracted IF can be determined using standard clinical assays. These assays require between 1 and 50 microliters of sample fluid. A Nanodrop® ND100 spectrophotometer capable of analyzing 2 μl volumes of solution can be used if the extracted IF volumes are insufficient for standard clinical assays. The IF biomarker concentrations can be correlated with levels found in whole blood or serum. The Human Metabolome Database (HMDB, www.hmdb.ca) can be used to understand the type and level of metabolites generally present in different kinds of biofluids, e.g., blood or cellular cytoplasm, where presence of a metabolite in more than one biofluid indicates a greater likelihood of presence in interstitial fluid. For instance, the HMDB entry for lactic acid (www.hmdb.ca/metabolites/HMDB00190) provides the presence of this metabolite in blood (e.g., at a concentration of 740-6400 μM in adults), cellular cytoplasm (e.g., 600-3500 μM), and cerebrospinal fluid (e.g., 450-3000 μM in adults). Lactic acid is present in arterial plasma at 600+/−70 μM and in interstitial fluid at 830+/−70 μM, both in adults. See M. Muller et al., Am. J. Physiol. Endo 271(6), E1003 (1996). The combination of the HMDB and literature searches can be used to identify useful biomarkers. These markers can be changed according to the need. Mass spectrometry can be used to directly analyze the protein and other biomarker composition of extracted IF for correlation determination.

Biomarkers availability in IF and correlation between IF and blood levels of these biomarkers can be used to guide the subsequent construction of specific sensor arrays. Several studies have shown equilibrium in glucose concentrations between IF and plasma using microneedles. See P. M. Wang et al., “Minimally invasive extraction of dermal interstitial fluid for glucose monitoring using microneedles,” Diabetes technology & therapeutics 7(1), 131 (2005). This finding suggests that biomarker levels present in the dermal IF may closely track those in serum, and that changes may be detectable earlier in IF. However, there have not been extensive studies of other relevant markers, including lactate, in IF that leverages the precise fluid extraction capabilities of microneedles. Previous studies used relatively large, 30-gauge needles and therefore had limited ability to control needle placement within specific layers of the dermis and epidermis. Optimized microneedles can be used to extract IF from precise, standardized depths in order to quantify levels of known markers of metabolic stress. Metabolites such as lactate and ketones accumulate rapidly, while other stress markers, such as cortisol, accumulate over time with repeated stress. The microneedle platform can incorporate different markers of stress to enable detection of acute, intermittent, and long-term stress. For instance, a common test for heart disease is the stress test, where a patient performs increasingly intense physical activity during continuous cardiac monitoring. A test similar to this with the detection platform can show correlations between biomarkers and vital signs (e.g. heart rate, blood pressure, respiratory rate). The sensor model can be used to create an integrated, multiplexed, autonomous on-body sensor array for known and emerging biomarkers.

A wide array of biomarkers from stress hormones (cortisol and adrenaline) to endogenous opioids (endorphins and enkephalins) can report on overall physiologic stress. For example, lactate can be used as a model system to define the correlation between IF and plasma biomarker concentrations for a cohort under metabolic stress. Lactate concentrations in the IF can track lactate concentrations in venous blood. Changes in IF lactate concentration can precede changes in venous blood concentration. The correlation between IF and blood lactate in cohorts undergoing a stress test can be quantified, demonstrating feasibility for continuous, non-invasive physiological monitoring with a microneedle array. Once the time correlation of venous and IF lactate is understood, and the stability of IF analysis through microneedle extraction is optimized, monitoring of other biomarkers is straightforward.

### Sensing Transducers to Monitor IF Biomarkers and Assess Levels of Biomarkers in Human Subjects Undergoing Physical Exertion with Focus on Lactate as a Model

Electrode arrays can be used as a sensing platform. Development of a sensitive electrode transducer requires knowledge of what concentrations the biomarkers exist in IF, which determines the analytical linear ranges in which the sensors operate. Also, knowledge of what other components are present in the interstitial fluid matrix is necessary to optimize the transducer to avoid detection of potential interfering species. Previously fabricated electrode transducers can be tailored to detect specific biomarkers. Various biomarkers may require separate electrode materials (e.g. gold, porous carbon, carbon paste) depending on their inherent electroactivity. Electrode transducers have previously been integrated with hollow polymeric microneedles for the ex-vivo detection of ascorbic acid and peroxide, potassium, and the simultaneous detection of glucose, lactate, and pH. See P. R. Miller et al., Biomicrofluidics 5, 013415 (2011); P. R. Miller et al., “Microneedle-Based Transdermal Sensor for On-Chip Potentiometric Determination of K+” Advanced healthcare materials (2013); and P. R. Miller et al., “Multiplexed microneedle-based biosensor array for characterization of metabolic acidosis,” Talanta 88, 739 (2012). The electrode transducers in these cases were placed either inside or directly underneath the microneedles, a configuration which is unlikely to enable long-term, repeat measurement of analytes of interest. To circumvent these problems, the microneedle device of the present inventions can extract interstitial fluid to be run over downstream electrode arrays, was shown in FIG. 1(a).

For example, electrode transducers can be of a size and geometry that are compatible with microneedle array IF delivery and that operate in the analytical range for lactate. IF lactate measurements described above can be used to determine the proper analytical range. (The analytical range is approximately 0.5-5 mM). The electrode transducers can remain stable over a time period of several hours and can perform continuous lactate monitoring with minimal drift. An electrode array of the geometry needed for lactate detection can be a multiplexed, integrated sensor. The stability of the sensor enables continuous lactate measurements over a period of hours to days.

The present invention has been described as a transdermal diagnostic device for in vivo extraction of interstitial fluid using hollow microneedles. It will be understood that the above description is merely illustrative of the applications of the principles of the present invention, the scope of which is to be determined by the claims viewed in light of the specification. Other variants and modifications of the invention will be apparent to those of skill in the art.

We claim:

1. An interstitial fluid extraction device, comprising:
an array of hollow microneedles adapted to penetrate the skin of a human or animal, wherein the skin comprises at least one biomarker in an interstitial fluid;
an injector for injecting saline solution into the skin through the array of hollow microneedles to mix with the interstitial fluid; and
a microfluidic chip adapted to extract the interstitial fluid and the injected saline solution mixture through the hollow microneedles and collect the at least one biomarker in a sample reservoir.

2. The device of claim 1, wherein the at least one biomarker comprises cortisol, a ketone, TNF-α, glutamine, glutamate, interleukin-6, testosterone, thyroid hormone, human growth hormone, insulin, glucose, adrenaline, or neuropeptide Y.

3. The device of claim 1, wherein the at least one biomarker comprises lactate.

4. The device of claim 1, wherein a concentration of the at least one biomarker in the interstitial fluid correlates with a concentration of the at least one biomarker in the blood plasma of the human.

5. The device of claim 1, further comprising a spectrophotometer for analyzing the at least one biomarker in the extracted interstitial fluid.

6. The device of claim 1, further comprising an electrode transducer for sensing the at least one biomarker in the extracted interstitial fluid.

7. The device of claim 1, wherein the hollow microneedles have a bore opening on the side of each microneedle.

8. The device of claim 7, wherein the bore opening is on the side to the middle third of each microneedle.

9. The device of claim 1, wherein the hollow microneedles each have an aspect ratio between 2 and 5.

10. The device of claim 1, wherein the hollow microneedles each have a base of between 300 and 500 microns.

11. The device of claim 1, wherein the hollow microneedles further comprise a coating to control hydrophilicity and promote fluid flow through the lumen of the hollow microneedle.

12. A method for extracting interstitial fluid from a human, comprising:
providing an interstitial fluid extraction device, the device comprising:
an array of hollow microneedles adapted to penetrate the skin of a human or animal, wherein the skin comprises at least one biomarker in an interstitial fluid,
an injector for injecting saline solution into the skin through the array of hollow microneedles to mix with the interstitial fluid, and
a microfluidic chip adapted to extract the interstitial fluid and the injected saline solution mixture through the hollow microneedles and collect the at least one biomarker in a sample reservoir;
injecting saline solution into the skin through the array of hollow microneedles to mix with the interstitial fluid; and
extracting the interstitial fluid and injected saline solution mixture through the array of hollow microneedles and collecting the at least one biomarker in the sample reservoir in the microfluidic chip.

13. The method of claim 12, wherein the at least one biomarker comprises cortisol, a ketone, TNF-α, glutamine, glutamate, interleukin-6, testosterone, thyroid hormone, human growth hormone, insulin, glucose, adrenaline, or neuropeptide Y.

14. The method of claim 12, wherein the at least one biomarker comprises lactate.

15. The method of claim 12, wherein a concentration of the at least one biomarker in the interstitial fluid correlates with a concentration of the at least one biomarker in the blood plasma of the human.

16. The method of claim 12, further comprising a spectrophotometer for analyzing the at least one biomarker in the extracted interstitial fluid.

17. The method of claim 12, further comprising an electrode transducer for sensing the at least one biomarker in the extracted interstitial fluid.

18. The method of claim 12, wherein the hollow microneedles have a bore opening on the side of each microneedle.

19. The method of claim 18, wherein the bore opening is on the side to the middle third of each microneedle.

20. The method of claim 12, wherein the hollow microneedles each have an aspect ratio between 2 and 5.

21. The method of claim 12, wherein the hollow microneedles each have a base of between 300 and 500 microns.

22. The method of claim 12, wherein the hollow microneedles further comprise a coating to control hydrophilicity and promote fluid flow through the lumen of the hollow microneedle.
